(19) United States
(12) Patent Application Publication
(10) Pub. No.: US 2019/0192618 A1
(43) Pub. Date: Jun. 27, 2019

(54) SPR741 HUMAN PHARMACOKINETICS AND EFFICACIOUS DOSE

(71) Applicant: SPERO POTENTIATOR, INC., Cambridge, MA (US)

(72) Inventors: Scott COLEMAN, Cambridge, MA (US); Prathap N. SHASTRI, Cambridge, MA (US)

(73) Assignee: SPERO POTENTIATOR, INC., Cambridge, MA (US)

(21) Appl. No.: 16/301,355

(22) PCT Filed: May 15, 2017
(86) PCT No.: PCT/US2017/032669

§ 371 Date: Nov. 13, 2018

Related U.S. Application Data
(60) Provisional application No. 62/336,208, filed on May 13, 2016.
(60) Provisional application No. 62/347,174, filed on Jun. 8, 2016.

(51) Int. Cl. A61K 38/12; A61K 9/00; A61K 31/7052; A61K 31/7048; A61K 31/575; A61K 31/351; A61K 31/46; A61K 31/496; A61K 31/407; A61K 38/14

(52) U.S. Cl. CPC A61K 38/12; A61K 9/0019; A61K 31/7052; A61K 31/7048; A61K 38/14; A61K 31/351; A61K 31/46; A61K 31/496; A61K 31/407; A61K 31/575

(57) ABSTRACT

This disclosure provides a method of treating a bacterial infection in a human patient comprising by administering a therapeutically effective dose of SPR741, a polymyxin analog, in combination with a therapeutically effective amount of an antibiotic other than SPR741. The disclosure establishes 100 mg to 500 mg of SPR741, administered 2 to 4 times daily as the effective amount of SPR741 for co-administration with a therapeutically effective amount of a second antibiotic. The disclosure also provides a method of treating a bacterial infection comprising administering 40 mg/kg patient weight/day or less and preferably 5 mg/kg patient weight/day or less of SPR741 in combination with a therapeutically effective amount of a second antibiotic.

### CROSS REFERENCE TO RELATED APPLICATIONS

This application claims priority from U.S. Provisional application 62/336,208, filed May 13, 2016 and 62/347,174 filed Jun. 8, 2016, both of which are incorporated by reference in their entirety.

### BACKGROUND

Gram-negative bacteria cause more than 40% of all septicemic infections and many of the Gram-negative bacteria are resistant to multiple antibiotics. Gram-negative bacteria possess lipopolysaccharide as a component of the outer membrane, which inhibits the diffusion of many antibacterial agents deeper into the cell, where their ultimate targets are located. Many antibacterial agents effective against Gram-positive bacteria lack activity against Gram-negative bacteria.

Polymyxins are a group of closely related antibiotic substances produced by strains of Paenibacillus polymyxa and related organisms. These cationic drugs are relatively simple peptides with molecular weights of about 1000. Polymyxins, such as polymyxin B, are decapeptide antibiotics, i.e., they are made of ten (10) aminoacyl residues. They are bactericidal and especially effective against Gram-negative bacteria such as Escherichia coli and other species of Enterobacteriaceae, Pseudomonas, Acinetobacter baumannii, and others. However, polymyxins have severe adverse effects, including nephrotoxicity and neurotoxicity. These drugs thus have limited use as therapeutic agents because of high systemic toxicity.

Polymyxins were widely used in the therapy of serious infections caused by those bacteria since their discovery in the 1950's, but because of the toxicity, their use was largely abandoned in the 1970's when newer, better tolerated antibiotics were developed. The recent emergence of multiresistant strains of Gram-negative bacteria has resulted in many of the less toxic antibiotics losing their effectiveness against Gram negative bacteria. Polymyxins have maintained their effectiveness against these emergent multiresistant strains of Gram-negative bacteria. Accordingly, polymyxins have been recalled to the therapeutic arsenal, although, due to their toxicity, they are considered a therapeutic of last resort. Their systemic (i.e., non-topical) use is, however, largely restricted to the therapy of life-threatening infections caused by multiply resistant strains of Pseudomonas aeruginosa and A. baumannii as well as by carbapenem-resistant Enterobacteriaceae.

SPR741, Pub Chem ID 53323381, as the structure Acetyl-Thr-dSer-cy[Dab-Dab-dPhe-Leu-Dab-Dab-Thr], where Dab is Acetyl-Thr-dSer-cy[Dab-Dab-dPhe-Leu-Dab-Dab-Thr], where Dab is an α,γ-diamino-n-butyryl residue and cy is cyclic, and is also shown below as a chemical structure.

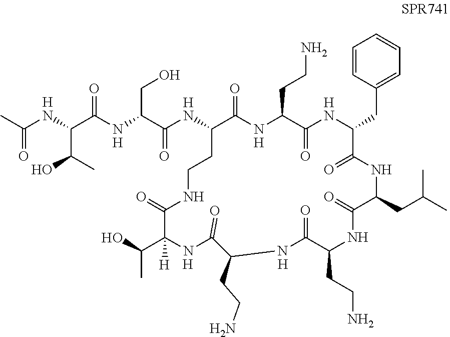

SPR741 has previously been shown to increase the sensitivity of certain bacteria to Mupirocin, Azithromycin, Fusidic Acid, and Vancomycin. SPR741 permeabilizies the outer membrane of Gram negative bacteria thus granting antibiotics that would otherwise be excluded access to their targets when administered in combination with SPR741.

### SUMMARY

This disclosure includes a method of treating a bacterial infection in a human patient comprising administering 100 mg to 500 mg of SPR741 2 to 4 times daily in combination with therapeutically effective amount of an antibiotic.

The disclosure includes a dosage form comprising 100 mg to 500 mg SPR741 and a carrier.

### BRIEF DESCRIPTION OF THE DRAWINGS

FIG. 1. Simulated concentration vs. time profiles in humans following a single 60 minute IV infusion of SPR741 at 100, 200, 300, 400, and 800 mg.

### DETAILED DESCRIPTION

Determination of the efficacious human dose of SPR741 was made using a three species allometric scaling analysis. The projected human PK profile and efficacious dose of SPR741 are consistent with potential partner antibiotics. A therapeutically effective amount of a pharmaceutical composition/combination is an amount effective, when administered to a subject, to provide a therapeutic benefit, such as to decrease the morbidity and mortality associated with bacterial infection and/or effect a cure. In certain circumstances a subject suffering from a microbial infection may not present symptoms of being infected. Thus a therapeutically effective amount of a compound is also an amount sufficient to significantly reduce the detectable level of microorganism in the subject's blood, serum, other bodily fluids, or tissues. The disclosure also includes, in certain embodiments, using compounds of the disclosure in prophylactic treatment and therapeutic treatment. In the context of prophylactic or preventative treatment, a “therapeutically effective amount” is an amount sufficient to significantly decrease the incidence of or morbidity and mortality associated with bacterial infection. For example, prophylactic treatment may be administered when a subject is known to be at enhanced risk of bacterial infection, such cystic fibrosis or ventilator patients. A significant reduction is any detectable negative change that is statistically significant in a standard parametric test of statistical significance such as Student's T-test, where p<0.05.

“Pharmaceutical compositions” are compositions comprising at least one active agent, such as a SPR741, and at least one other substance, such as an antibiotic, or a carrier. Pharmaceutical compositions meet the U.S. FDA's GMP (good manufacturing practice) standards for human or non-human drugs. The term “carrier” applied to pharmaceutical compositions/combinations of the disclosure refers to a diluent, excipient, or vehicle with which an active compound is provided.

The pharmaceutical compositions of the disclosure include ocular, oral, nasal, transdermal, topical with or without occlusion, intravenous (both bolus and infusion), inhalable, and injection (intraperitoneally, subcutaneously, intramuscularly or parenterally) formulations. The composition may be in a dosage unit such as a tablet, pill, capsule, powder, granule, liposome, sterile ocular solution, parenteral solution or suspension, metered aerosol or liquid spray, drop, ampoule, auto-injector device, or suppository; for administration ocularly, orally, intranasally, sublingually, parenterally, or rectally, or by inhalation or insufflation.

The dosage form containing the composition of the disclosure contains an effective amount of the active agent necessary to provide a therapeutic effect by the chosen route of administration. The composition may contain from about 5,000 mg to about 0.5 mg (preferably, from about 1,000 mg to about 0.5 mg) of a compound of the disclosure or salt form thereof and may be constituted into any form suitable for the selected mode of administration. The dosage form may be formulated for immediate release or controlled release, including delayed release or sustained release. The pharmaceutical composition includes SPR741 and at least one direct acting antibiotic (a compound efficacious for killing pathogenic bacteria in vivo) for example, retapamulin, telithromycin, aztreonam.

SPR741 has been assessed for inhibition of the hERG mediated potassium current at concentrations up to 300 μg/mL in a hERG assay using standard procedures. SPR741 was assessed for cardiopulmonary effects and general toxicity based on mortality, clinical observations, body weight, body temperature, blood pressure (systolic, diastolic, and mean arterial), heart rate, the electrocardiogram (QRS duration and the RR, PR, and QT intervals) and respiratory function (respiratory rate, tidal volume, and minute volume) in a cardiopulmonary safety pharmacology study in monkeys. SPR741 has also been assessed for central nervous system (CNS) effects based on reaction to environmental stimuli, involuntary or stereotypic behavior, gait, brachiation, posture, grasp, activity level, balance, conjugate movement, position, and pupil reactivity as part of the GLP 14-day repeat dose toxicology study in monkeys.

In the hERG study, superfusion of SPR741 produced no meaningful change in mean hERG-mediated potassium currents at concentrations up to 300 μg/mL. Due to the lack of effect by SPR741, an IC⁵⁰ could not be calculated. In the cardiopulmonary study, there were no SPR741-related clinical signs, changes in body weight, changes in body temperature, changes noted in systolic, diastolic, or mean arterial blood pressures, changes noted in heart rate, changes noted in the RR interval, PR interval, uncorrected QT interval, QRS duration, or QTc, or changes noted in respiratory rate, tidal volume, or minute volume. In the CNS study, there were no SPR741 changes noted for any of the neurological assessments.

### Specific Embodiments

The disclosure includes a method of treating a bacterial infection in a human patient comprising administering 100 mg to 500 mg of SPR741 2 to 4 times daily in combination with therapeutically effective amount of an antibiotic.

The disclosure includes methods in which:

(1) 200 mg to 400 mg of SPR741 are administered 3 times daily.

(2) The bacterial infection is a Gram negative bacterial infection.

(3) The bacterial infection is an E. coli infection, a Klebsiella pneumoniae infection, an Acinetobacter baumannii infection, a Pseudomonas aeruginosa, a Neisseria gonorrhoeae infection, or a Yersinia pestis infection.

(4) The infection is a Mycobacterium an E. coli infection, a Klebsiella pneumoniae infection, or an Acinetobacter baumannii infection.

(5) The antibiotic is azithromycin, clarithromycin, fusidic acid, mupirocin, retapamulin, rifampicin, telithromycin, meropenem, mupirocin, azithromycin, or vancomycin.

The disclosure includes a pharmaceutical dosage form comprising 100 mg to 500 mg SPR741 or 200 mg to 400 mg and a carrier.

(6) A method of treating a bacterial infection in a human patient comprising administering 40 mg/kg patient weight/day or less, or 30 mg/kg patient weight/day or less, or 20 mg/kg patient weight/day or less, or 10 mg/kg patient weight/day or less, or 5 mg/kg patient weight/day or less, of SPR741 in combination with a therapeutically effective amount of a second antibiotic.

(7) The method of Embodiment (6) where the 40 mg/kg patient weight/day or 30 mg/kg patient weight/day or 20 mg/kg patient weight/day or 10 mg/kg patient weight/day or 5 mg/kg patient weight/day of SPR741 is administered in doses given 2 to 4 times daily, or 3 times daily.

(8) The method of Embodiment (6) or (7), wherein the bacterial infection is a Gram negative bacterial infection.

(9) The method of Embodiment (6) or (7), wherein the bacterial infection is an E. coli infection, a Klebsiella pneumoniae infection, an Acinetobacter baumannii infection, a Pseudomonas aeruginosa, a Neisseria gonorrhoeae infection, or a Yersinia pestis infection.

(10) The method of any one of Embodiments (6) to (9), wherein the second antibiotic is azithromycin, clarithromycin, fusidic acid, mupirocin, retapamulin, rifampicin, telithromycin, meropenem, mupirocin, azithromycin, or vancomycin.

(11) The method of any one of Embodiments (6) to (10), wherein the SPR741 is administered injectably, orally, or intravenously.

In certain embodiments the disclosure includes a pharmaceutical composition in which:

(1) The dosage form is an injectable or intravenous formulation.

(2) The dosage form is an oral dosage form.

(3) The oral dosage form additionally comprises an antibiotic.

(4) The antibiotic is azithromycin, clarithromycin, fusidic acid, mupirocin, retapamulin, rifampicin, telithromycin, meropenem, mupirocin, azithromycin, or vancomycin.

### EXAMPLES

The following abbreviations are used in the examples:
AUC Area Under Curve
CL Clearance
Cₘₐₓ Maximum observed concentration
h hour(s)
HED Human Equivalent Dose
PK Pharmacokinetic
T_{1/2} Terminal half-life
V_{d} Volume of distribution
W Body Weight
NOAEL No Observed Adverse Effects Level

### Example 1

### Prediction of Human PK Parameters for SPR741 Via Allometric Scaling

Human pharmacokinetic (PK) parameters for SPR741 were estimated using both fixed and floating exponent allometric scaling methods. SPR741 PK data from mouse, rat, and monkey studies were fit to a 1-compartmental model and the same model was used for human simulations. PK analysis was performed using Phoenix® WinNonlin® Version 6.3 (Pharsight Corp. [Mountain View, Calif.]). Mean plasma concentrations of SPR741 were used to derive PK parameters from preclinical studies.
Linear elimination PK was assumed to derive PK parameters from animal studies and for human simulations. The standard species body weights were assumed to perform allometric scaling purposes. For scaling, mouse, rat, monkey, and human weight were assumed to be 0.02, 0.25, 5, and 70 kg, respectively. PK data from only male animals were available for scaling.

A one-compartment model was fitted to mean SPR741 PK profiles from mouse, rat, and monkey PK studies. A MixRatio (additive and multiplicative) weighting was applied for all PK modeling. Two sets of scaled human PK parameters for SPR741 that yielded a wide range of estimates were used to calculate Human Equivalent Dose (HED) and to simulate SPR741 concentrations in healthy subjects after a single one-hour IV infusion dose.

Allometric scaling was performed using both fixed (1) and floating (2) exponent scaling method using the following equations:

CLₕᵤₘₐₙ=CLₐₙᵢₘₐₗ·(Wₕᵤₘₐₙ/Wₐₙᵢₘₐₗ)^{0.75} (1)

PK Parameter=a*(W)b (2)
Within formula (1) CL is clearance and W is body weight. The exponent was fixed at 0.75 for clearance parameters and 1 for volume of distribution parameters.

In the floating exponent allometric method (2) a linear regression was performed for PK parameters (from mouse, rat, and monkey) against body weight. The slope, a, and intercept, b, were used to calculate PK parameters, such as clearance. Standard body weights were used in the calculations, 0.02 kg for mouse, 0.25 kg for rat, 5 kg for monkey, and 70 kg for human.

Human clearance and half-life estimates for SPR741 from various allometric approaches ranged from 51 to 103 mL/h/kg and 2.2 to 2.4 hours, respectively. Efficacy studies in mice with SPR741 demonstrated pharmacological activity with an AUC ranging from 2 to 60 μg*h/mL. The projected human efficacious dose for SPR741 ranges from approximately 100 to 400 mg administered three times per day using the scaled clearance and the AUC that demonstrated efficacy in the mouse studies. Human PK simulations were performed after a single IV infusion administration of SPR741 with the AUC ranging from 14 to 28, 28 to 56, 55 to 112, and 110 to 225 μg*h/mL at 100, 200, 400, and 800 mg, respectively. Pharmacokinetic parameters estimated using the fixed exponent and floating exponent allometric approaches are provided in Table 1. A 70 kg human weight was used in all models.

TABLE 1

| | PK Data Used | CL | V_{d} | T_{1/2} |
|---|---|---|---|---|
| Method | for Scaling | (mg/h/kg) | (mL/kg) | (h) |
| Fixed | Mouse | 57.5 | 179 | 2.16 |
| Fixed | Rat | 103 | 345 | 2.31 |
| Fixed | Monkey | 70.8 | 238 | 2.33 |
| Floating | Rat and Monkey | 50.8 | 172 | 2.34 |
| Floating | Mouse, Rat, | 89.7 | 315 | 2.44 |
| | Monkey | | | |

### Example 2

### Pharmacologic Human Equivalent Dose (HED) Projections

The HED was based on efficacy data from mouse thigh studies with SPR741 and rifampin with an AUC value of 60 μg*h/mL. Pharmacologic HED was then calculated using the equation below:

Dose=Cl*AUC (3)

Dose=Pharmacologic HED, Cl=estimated human clearance obtained from allometric scaling, AUC=60 μg*h/mL.

Projected Human Equivalent Doses obtained using the fixed and floating allometric scaling models are shown in Table 2.

TABLE 2

| | | CL | Vd | T_{1/2} | HED |
|---|---|---|---|---|---|
| Method | PK Data Used | (mL/h/kg) | (mL/kg) | (h) | (mg) |
| Fixed | Mouse | 57.5 | 179 | 2.16 | 241 |
| Fixed | Rat | 103 | 345 | 2.31 | 434 |
| Fixed | Monkey | 70.8 | 238 | 2.33 | 297 |
| Floating | Rat and Monkey | 50.8 | 172 | 2.34 | 213 |
| Floating | Mouse, Rat, and | 89.7 | 315 | 2.44 | 377 |
| | Monkey | | | | |

### Example 3

### Human PK Simulation

Single dose simulations were done using the low clearance estimate and high clearance estimate obtained from allometric scaling. A 1-compartmental model that was used to fit animal data was used for human simulations after a single IV infusion dose (Table 3). Day 1 data was unavailable for rat. Only 2-3 measurable time points were used in the mouse PK data.

TABLE 3

| | Dose levels | Data used | CL | V_{d} |
|---|---|---|---|---|
| Species | (mg/kg) | (mg/kg) | (mL/h/kg) | (mL/kg) |
| Mouse | 1, 3, and 10 | Day 1 | 442 | 179 |
| Rat | 1.67, 5, and 10 | Day 7 | 423 | 345 |
| Monkey | 4, 10, and 20 | Day 1 | 137 | 238 |

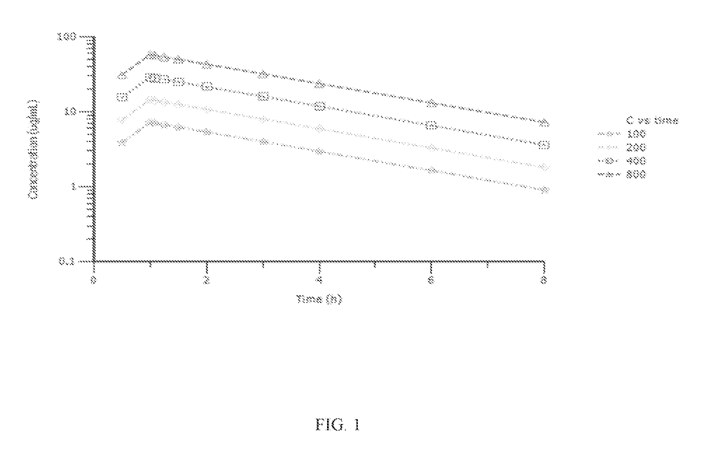

A non-compartmental analysis was done using concentration time values from the simulations to obtain estimated human PK parameters at a dose of 100, 200, 400, and 800 mg/kg. Table 4 provides the projected clinical AUC and Cₘₐₓ in humans derived from a 60-minute single IV infusion of SPR741 at 100, 200, 400, and 800 mg/kg. This data is also shown in graph form in FIG. 1.

TABLE 4

| Dose | AUC | Cₘₐₓ | T_{1/2} |
|---|---|---|---|
| (mg) | (μg · hr/mL) | (μg/mL) | (h) |
| 100 | 14-28 | 4-7 | 2.3-2.4 |
| 200 | 28-56 | 7-14 | 2.3-2.4 |
| 400 | 55-112 | 14-28 | 2.3-2.4 |
| 800 | 110-225 | 28-58 | 2.3-2.4 |

Simulated SPR741 Concentration vs. Time data for humans following a single IV infusion administration of SPR741 at 100, 200, 400, and 800 mg/kg is for two simulations are provided in Tables 5 and 6. Time points are provided with respect to the start of the IV infusion.

TABLE 5

Dose (mg)

| | | | |
|---|---|---|---|
| 100 | 200 | 400 | 800 |

| | |
|---|---|
| Time (hr) | Concentration (μg/mL) |

| | | | | |
|---|---|---|---|---|
| 0.5 | 1.92 | 3.84 | 7.68 | 15.4 |
| 1 | 3.57 | 7.14 | 14.3 | 28.6 |
| 1.08 | 3.48 | 6.97 | 13.9 | 27.9 |
| 1.25 | 3.31 | 6.63 | 13.3 | 26.5 |
| 1.5 | 3.08 | 6.15 | 12.3 | 24.6 |
| 2 | 2.65 | 5.30 | 10.6 | 21.2 |
| 3 | 1.96 | 3.93 | 7.85 | 15.71 |
| 4 | 1.46 | 2.91 | 5.82 | 11.7 |
| 6 | 0.80 | 1.60 | 3.20 | 6.4 |
| 8 | 0.44 | 0.88 | 1.76 | 3.52 |

TABLE 6

Dose (mg)

| | | | |
|---|---|---|---|
| 100 | 200 | 400 | 800 |

| | |
|---|---|
| Time (hr) | Concentration (μg/mL) |

| | | | | |
|---|---|---|---|---|
| 0.5 | 3.87 | 7.74 | 15.5 | 31.0 |
| 1 | 7.21 | 14.42 | 28.8 | 57.7 |
| 1.08 | 7.04 | 14.1 | 28.1 | 56.3 |
| 1.25 | 6.70 | 13.4 | 26.8 | 53.6 |
| 1.5 | 6.22 | 12.4 | 24.9 | 49.73 |
| 2 | 5.36 | 10.7 | 21.4 | 42.9 |
| 3 | 3.98 | 7.97 | 15.9 | 31.9 |
| 4 | 2.96 | 5.92 | 11.8 | 23.7 |
| 6 | 1.64 | 3.27 | 6.54 | 13.1 |
| 8 | 0.90 | 1.81 | 3.92 | 7.23 |

### Example 7

### GLP 14 Day Repeat Dose Study of SPR741 in Sprague-Dawley Rats

SPR741 was assessed for potential toxicity in rats at dose levels of 5, 15, and 30 mg/kg/day via a one-hour infusion twice per day 12 hours apart for 14 consecutive days. Parameters assessed during the in-life phase of the study included weekly body weights, clinical observations, survival, food consumption, ophthalmology, clinical pathology, and urine and plasma toxicokinetics. At necropsy, gross observations were recorded, organ weights were measured, and specific tissues were collected. Histopathologic assessment was conducted on tissue sections stained with hematoxylin and eosin (H&E).

SPR741 in saline was administered twice daily at 5, 15, or 30 mg/kg/day approximately 12 hours apart at escalating doses for the first 3 days, followed by 14 consecutive days at the target dose level) via 1-hour intravenous infusion via indwelling catheters. The use of an escalation phase was due to the acute histamine and/or complement mediated reactions commonly seen in rats with a broad range of polymyxins. SPR741 exhibited dose dependent proportional systemic exposure following 14 days of repeated dosing (TABLE 7).

For toxicology assessment, all animals were observed twice daily for mortality and moribundity. Clinical examinations were performed daily, and detailed physical examinations were performed weekly. Ophthalmic examinations were performed during once prior to study initiation and once during the study. Clinical pathology parameters (including Blood Urea Nitrogen and Serum Creatinine) were analyzed for all animals assigned to the scheduled necropsies. Complete necropsies were conducted on all animals, and selected organs were weighed at the scheduled necropsies. Selected tissues were examined microscopically from all animals found dead or euthanized in extremis and from all animals in the control, 15, and 10 mg/kg/day groups at the scheduled necropsies. Gross lesions and infusion sites were examined from all animals in the low-dose group at the primary necropsy. Recovery was assessed in the mid-dose group and the control group following a 28-day non-dosing period. Following the 28-day non-dosing (recovery) period, complete necropsies were conducted on all animals, and selected organs were weighed at the scheduled necropsy. Selected tissues were examined microscopically from all animals.

For toxicokinetic assessment, blood samples were collected from the SPR741 dosed rats prior to dose administration, immediately following the end of dose administration (within 5 minutes), and at approximately 30 minutes and 1, 2, 4, and 12 hours (immediately prior to the second BID dose) post end of the first BID dose on study days 1 and 14. The plasma from these samples were analyzed for SPR741 using a validated bioanalytical method. TABLE 8 shows the changes observed for blood urea nitrogen, creatinine and kidney tubular regeneration.

The dose level of 5 mg/kg was well tolerated. Higher dose levels exhibited toxicity.

TABLE 7
Dose Proportional Systemic Exposure to SPR741

| Dose | | Cmax | AUC(0-24) |
|---|---|---|---|
| (mg/kg/day) | Study Day | (μg/ml) | μg*hr/mL |
| 5 | 1 | 3.4 | 8.2 |
| | 14 | 3.3 | 8.3 |
| 10 | 1 | 9.5 | 25.0 |
| | 14 | 6.7 | 20.3 |
| 15 | 1 | 14.9 | 47.4 |
| | 14 | 10.8 | 32.9 |

TABLE 8
Non-adverse findings for 5 mg/kg/day dose

| | % BUN | | |
|---|---|---|---|
| | increase | | |
| | relative | % | |
| Dose | to control | Control | |
| (mg/kg/day) | (mg/dL) | CRN | Renal Histopathology |
| 5 | — | — | 2/9 minimal tubular degeneration |
| | | | 7/9 mild tubular degeneration |
| | | | 2/9 minimal tubular necrosis |
| 10 | 34 | — | 1/10 minimal tubular degeneration |
| | | | 4/10 mild tubular degeneration |
| | | | 5/10 moderate tubular degeneration |
| | | | 5/10 minimal tubular necrosis |
| 15 | 59 | 59 | 7/7 moderate tubular degeneration |
| | | | 5/10 minimal tubular degeneration |

### Example 8

### A GLP 14 Day Repeat Dose Toxicology Study of SPR741 in Monkeys

SPR741 was assessed for toxicity in monkeys at dose levels of 20, 40, 60, and 80 mg/kg/day via a one-hour infusion three times per day (8 hours apart for 14 consecutive days. Parameters assessed during the in-life phase of the study included weekly body weights, clinical observations (14×/day during the dosing period), food consumption, neurological examinations, electrocardiography (ECG), ophthalmology, clinical pathology (hematology, coagulation, serum chemistry, urinalysis), and urine and plasma TK. At necropsy, gross observations were recorded, organ weights were measured, and specific tissues were collected. Histopathologic assessment was conducted on tissue sections stained with hematoxylin and eosin (H&E).

The NOAEL of SPR741 following 14 days of repeated three times per day one hour infusions in male and female cynomolgus monkeys was 40 mg/kg/day. There were no SPR741 related effects on food consumption, electrocardiography, or ophthalmology. The target organ of toxicity for SPR741 in monkeys is the kidney. SPR741 at dose levels of greater than 40 mg/kg/day was associated with adverse nephrotoxicity demonstrated by mild to moderate increases in blood urea nitrogen and serum creatinine. The mild to moderate increase in biomarkers of renal function were accompanied by pale kidneys, higher kidney weight, and histopatholgical changes in the kidney of tubular regeneration, degeneration/necrosis, casts and dilation. The nephrotoxicity was fully reversible following a 28-day recovery period. SPR741 was not associated with any neurological effects at any dose level including the highest dose tested of 80 mg/kg/day.

Conclusions: The target organ of toxicity for SPR741 in this study is the kidney. The toxicity is monitorable and reversible and therefore demonstrates criteria for advancement into Phase 1 studies.

### Example 9

### SPR741 GLP Safety and Pharmacology Studies in Cardiac, Pulmonary, and CNS Systems

SPR741 was assessed for inhibition of the hERG mediated potassium current at concentrations up to 300 μg/mL in a hERG assay using standard procedures. Briefly, HEK293 cells stably expressing the hERG potassium channel were superfused with SPR741 at concentrations up to 300 μg/mL. Currents were recorded via single cell patch clamp. Results of these studies are summarized in TABLE 10.

TABLE 10
Summary of hERG Current Inhibition with SPR741

| Concentration | Inhibition % Mean | SEM | N |
|---|---|---|---|
| Vehicle | 1.02 | 0.281 | 3 |
| 10 μg/mL | 0.08 | 0.332 | 3 |
| 30 μg/mL | −0.09 | 0.727 | 3 |
| 100 μg/mL | 1.62 | 0.825 | 3 |
| 300 μg/mL | 3.87 | 0.928 | 3 |
| 0.047 μg/mL Cisapride | 79.80 | 1.708 | 3 |

N = number of measure used to calculate the mean,
SEM = Standard Error of the Mean

SPR741 was assessed for cardiopulmonary effects and general toxicity based on mortality, clinical observations, body weight, body temperature, blood pressure (systolic, diastolic, and mean arterial), heart rate, the electrocardiogram (QRS duration and the RR, PR, and QT intervals) and respiratory function (respiratory rate, tidal volume, and minute volume) in a cardiopulmonary safety pharmacology study in monkeys at dose levels of 5, 10 and 20 mg/kg administered via a single one hour infusion. The monkeys in the study were telemeterized prior to study start. Data was collected in one minute intervals over a 24 hour period. The data was reported in 15 minute intervals over the first three hours. Results of the cardiovascular and pulmonary studies are shown in TABLE 11.

TABLE 11
Summary of Cardiovascular and Pulmonary Functional Assessments
with SPR741 Over Three Hours Post Infusion

| | Body | Mean Arterial | Heart | QTc | Respira- |
|---|---|---|---|---|---|
| Dose | Temperature | Blood Pressure | Rate | Interval | tory |
| (mg/kg/day) | (° C.) | (mm Hg) | (bpm) | (msec) | Rate |
| Vehicle | 38.8 | 100 | 1389 | 217 | 25 |
| 5 | 38.3 | 105 | 142 | 220 | 27 |
| 10 | 38.3 | 104 | 141 | 222 | 25 |
| 20 | 38.7 | 97 | 145 | 220 | 26 |

SPR741 was assessed for central nervous system (CNS) effects based on reaction to environmental stimuli, involuntary or stereotypic behavior, gait, brachiation, posture, grasp, activity level, balance, conjugate movement, position, and pupil reactivity as part of the GLP 14-day repeat dose toxicology study in monkeys at dose levels of 20, 40, 60, and 80 mg/kg/day. The neurological assessment was performed on study day 9 or 10 and compared to both pre-test data (study day −6 or −7) as well as the vehicle control. All CNS assessments were normal.

1. A method of treating a bacterial infection in a human patient comprising administering 100 mg to 500 mg of SPR741 2 to 4 times daily in combination with therapeutically effective amount of an antibiotic.

2. The method of claim 1, wherein the 200 mg to 400 mg of SPR741 are administered 3 times daily.

3. The method of claim 1, wherein the bacterial infection is a Gram negative bacterial infection.

4. The method of claim 1, wherein the bacterial infection is an E. coli infection, a Klebsiella pneumoniae infection, an Acinetobacter baumannii infection, a Pseudomonas aeruginosa, a Neisseria gonorrhoeae infection, or a Yersinia pestis infection.

5. The method of claim 1, wherein the infection is an E. coli infection, a Klebsiella pneumoniae infection, or an Acinetobacter baumannii infection.

6. The method of claim 1, wherein the antibiotic is azithromycin, clarithromycin, fusidic acid, mupirocin, retapamulin, rifampicin, telithromycin, meropenem, mupirocin, azithromycin, or vancomycin.

7. A pharmaceutical dosage form comprising 100 mg to 500 mg SPR741 and a carrier.

8. The dosage form of claim 7 comprising 200 mg to 400 mg SPR741.

9. The dosage form of claim 7, wherein the dosage form is an injectable or intravenous formulation.

10. The dosage form of claim 7, wherein the dosage form is an oral dosage form.

11. The oral dosage form of claim 10, additionally comprising an antibiotic.

12. The oral dosage form of claim 11, wherein the antibiotic is azithromycin, clarithromycin, fusidic acid, mupirocin, retapamulin, rifampicin, telithromycin, meropenem, mupirocin, azithromycin, or vancomycin.

13. The method of claim 2, wherein the bacterial infection is a Gram negative bacterial infection.

14. The method of claim 2, wherein the bacterial infection is an E. coli infection, a Klebsiella pneumoniae infection, an Acinetobacter baumannii infection, a Pseudomonas aeruginosa, a Neisseria gonorrhoeae infection, or a Yersinia pestis infection.

15. The method of claim 2, wherein the infection is an E. coli infection, a Klebsiella pneumoniae infection, or an Acinetobacter baumannii infection.

16. The method of claim 2, wherein the antibiotic is azithromycin, clarithromycin, fusidic acid, mupirocin, retapamulin, rifampicin, telithromycin, meropenem, mupirocin, azithromycin, or vancomycin.

17. The dosage form of claim 8, wherein the dosage form is an injectable or intravenous formulation.

18. The dosage form of claim 8, wherein the dosage form is an oral dosage form.
